(19) United States
(12) Patent Application Publication
(10) Pub. No.: US 2018/0042685 A1
(43) Pub. Date: Feb. 15, 2018

(54) MEDICAL DEVICE USER INTERFACE WITH STERILE AND NON-STERILE OPERATION

(71) Applicant: DENTAL WINGS INC., MONTREAL (CA)

(72) Inventors: JULIEN MARBACH, MONTREAL (CA); ROBIN PROVOST, MONTREAL (CA); JEAN-MARC PEROT, MONT-ROYAL (CA); MICHAEL RYNERSON, MONTREAL (CA)

(21) Appl. No.: 15/556,451

(22) PCT Filed: Mar. 7, 2016
(86) PCT No.: PCT/CA2016/050241

§ 371 Date: Sep. 7, 2017

Related U.S. Application Data
(60) Provisional application No. 62/129,775, filed on Mar. 7, 2015.

(51) Int. Cl. A61B 34/00; A61C 9/00; A61B 1/24

(52) U.S. Cl. CPC A61B 34/25; A61B 1/24; A61C 9/004; G06F 19/3406

(57) ABSTRACT

A touch screen interface of a medical or dental device coupled with a touchless interface to allow for sterile zone operator input, while still allowing for touch screen interaction for non-sterile zone operator input. In some embodiments, the touch screen interface usage is suspended while in a sterile zone mode, and resumed when the mode is switched to a non-sterile zone mode. In some embodiments, use of the touch screen interface while in a sterile zone mode of operation causes an alert message.

### FIELD OF THE INVENTION

The present application relates to medical and dental device user interfaces.

### BACKGROUND TO THE INVENTION

In dental and medical devices, operation of the devices is typically done in a sterile zone where instrumentation has been made sterile, and after use with a patient any reused instrumentation is made sterile again. Many such devices are connected by wire or wirelessly to computers or control devices that have interfaces or controls located outside of the sterile zone.

Conventionally, an assistant would use such interfaces instead of the surgeon. This allows the interface, located outside of the sterile zone, to be used at the command of the surgeon while remaining in the sterile zone.

It is known to remove the need for such an assistant by providing a touchless interface for the connected equipment located outside of the sterile zone. An example is described in U.S. Pat. No. 8,411,034 where a touchless interface is provided for medical systems. Examples of touchless interfaces include cameras and voice control.

In some cases, for example in the case of an oral scanning system, the user interface can be a touch screen computer, and the interface has many controls and functions offered to the user for manipulating the oral image data collected from the patient and for manipulating a patient's dental model data, in additional to controls used during data collection in interaction with the patient.

### SUMMARY OF THE INVENTION

Applicant has discovered that a touch screen interface of a medical or dental device can be coupled with a touchless interface to allow for sterile zone operator input, while still allowing for touch screen interaction for non-sterile zone operator input. In some embodiments, the touch screen interface usage is suspended while in a sterile zone mode, and resumed when the mode is switched to a non-sterile zone mode. In some embodiments, use of the touch screen interface while in a sterile zone mode of operation causes an alert message.

There is disclosed an interface for controlling a medical device comprising a sterile mode of operation and a non-sterile mode of operation. The interface comprises a screen on which at least one control object for controlling the medical device is displayed, a touch screen interface for manipulating the at least one control object, and a touchless interface for manipulating the at least one control object, wherein the touch screen interface is enabled when in the non-sterile mode of operation and disabled when in the sterile mode of operation.

There is also disclosed a method for controlling a medical device comprising a screen, at least part of the medical device for use in both a sterile-zone and a non-sterile zone. The method comprises displaying at least one control object for controlling the medical device on the screen, providing both a touch screen interface and touchless interface for manipulating the at least one control object, disabling the touch screen interface when the part is within the sterile zone, and enabling the touch screen interface when the part is within the non-sterile zone.

Additionally, there is disclosed a dental device comprising a handheld intraoral scanner comprising at least one camera, a controller for sending commands to the controller and storing images captured by the handheld intraoral scanner, a communication link between the handheld intraoral scanner and the controller for transmitting images captured by the intraoral scanner to the controller and transmitting commands from the controller to the handheld intraoral scanner, a screen coupled to the controller and on which at least one control object is displayed, a touch screen interface for manipulating the at least one control object, and a touchless interface for manipulating the at least one control object, wherein the touch screen interface is enabled when the in the non-sterile mode of operation and disabled when in the sterile mode of operation and further wherein the controller generates and sends the commands to the handheld intraoral scanner in response to manipulation of the control object by one of the touch screen interface, the touchless interface and combinations thereof.

### BRIEF DESCRIPTION OF THE DRAWINGS

The embodiments will be better understood by way of the following detailed description with reference to the appended drawings, in which:

FIG. 1 is a block diagram of an intraoral scanner system having a touch screen interface for data acquisition and dental arch model building that includes a touchless interface and a sterile zone mode controller.

### DETAILED DESCRIPTION OF THE ILLUSTRATIVE EMBODIMENTS

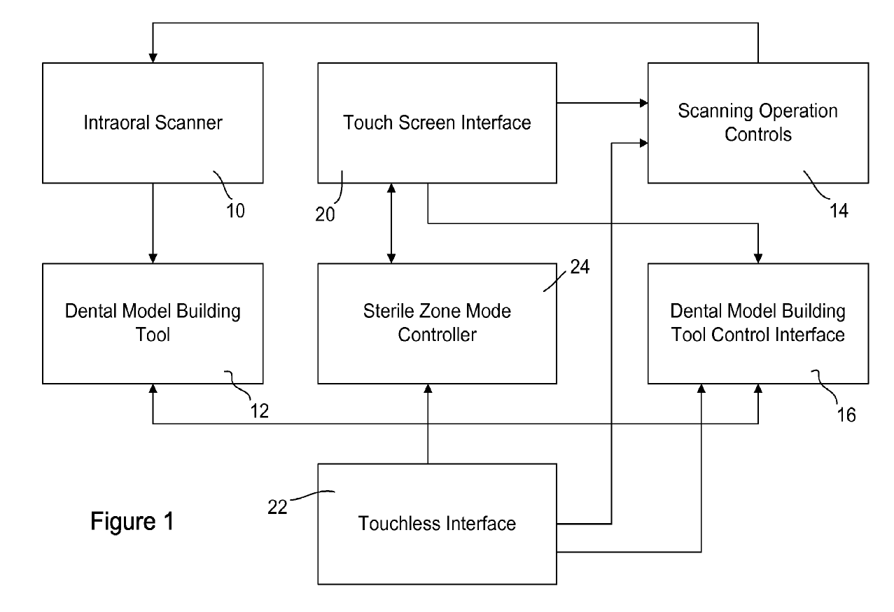

FIG. 1 illustrates schematically the components of an intraoral scanner system according to one embodiment chosen to illustrate an example of an embodiment. It will be appreciated that various modifications are possible.

Intraoral scanner 10 is a handheld device as is known in the art. The medical device can take the form of a variety of dental or surgical devices used within a sterile zone with a patient. Communication of scan data by wireless or wired communication is provided between the scanner 10 and a dental model building tool 12. Such a tool is also known in the art, and when the device 10 is not an oral scanner, the nature of the complementary tool 12 is likewise different. The tool 12 is typically implemented on an apparatus not located within the sterile zone.

The scanner 10 has a controller 14 that can be integrated into the scanner 10 or part of an apparatus located outside of the sterile zone. The controller 14 is known in the art. The controller 14 allows the user to send commands to the scanner in order to control the scanner for scanning operations. In the case that the device 10 is not an oral scanner, the controller 14 likewise controls different functions of the device 10.

The tool 12 has a controller 16 that is typically provided on the same apparatus as the tool 12. Illustratively, the controller 16 provides the model editing tools in the form of control objects used by the user for adjusting or correcting the dental arch model. When the tool 12 is not a dental model building tool, the controller 16 likewise provides different interface controls in the form of control objects which can be selected by the user, for example using a cursor. Illustratively, the editing tools/control objects are graphical elements displayed on the screen comprising, for example, one or more of a widget, a check box, a scroll bar, a radio button, a drop down box, a list box, a menu item, a button, spinner, a slider, a hyperlink, and the like and combinations thereof.

The controllers 14 and 16, in the embodiment of FIG. 1, obtain their user input and provide user feedback through a touch screen interface 20. It will be appreciated that such an interface can comprise a display with a painting device, such as a mouse or touch pad, and also optionally an external keyboard.

The touch screen interface can be used in the sterile zone if the screen is properly prepared, for example by wrapping with sterile plastic wrap. However, this requires an extra preparation step, and once the interface has been sterilized, there can be no contact with a person from outside of the sterile zone. This inconvenience is overcome by providing an additional touchless interface 22.

The touchless interface 22 can comprise a camera system, as is known in the art, for detecting gestures, for example gestures made in front of touch screen interface 20. This exemplary combination allows the user to use a single display device associated with the touch screen interface 20 and to use the same screen-based controls while simply making gestures to use the controls without actually contacting the touch screen interface 20 that would be outside of the sterile zone.

The touchless interface 22 can alternatively comprise a foot-controller mouse or trackball, as is known in the art, since in dental and surgical operations, the feet and ground are usually outside of the sterile zone and do not come into contact with the dentists' or surgeons' hands inside of the sterile zone. A foot-controlled mouse or trackball can be used to control a pointer and clicking device for manipulating the same controls as are provided on the touch screen interface 20. Likewise, the display used in the touch screen interface 20 can be also used as the display for the touchless interface 22.

The user interface for controllers 14 and/or 16 can be different for the touch screen interface 20 than for the touchless interface 22, or they can be the same. The touchless interface 22 can be simplified given the more limited user interaction of the interface, whether it be as a result of gesture recognition, voice commands or the use of foot controls.

The sterile zone mode controller 24 is a component added to the system of FIG. 1 that manages switching between the touch screen interface 20 and the touchless interface 22. While it is possible to have both interfaces active simultaneously, this requires trusting that a user within the sterile zone will abstain from using the touch screen interface 20 instead of using the touchless interface 22.

Controller 24 can serve a first purpose of reminding the user as to the mode of operation. In this configuration, a user input using the touch screen interface 20 or the touchless interface 22 signals to the controller 24 that the system will be henceforth used in a “sterile mode”. At this point, the display screen of the touch screen interface 20 can display a message, such as “sterile mode active, please put on gloves” as a reminder to the dentist or dental technician (or other medical professional) to put on sterile gloves. This message can remain on the display and/or the display can change a border or background color to provide a reminder of the mode of operation in which the touchless interface 22 is to be used without using the touch screen interface 20.

Controller 24 can end the sterile mode by receiving a user input from either the touch screen interface 20 or the touchless interface 22. At this point, the display screen of the touch screen interface 20 can display a message, such as “sterile mode ended, please remove gloves” as a reminder to the dentist or dental technician (or other medical professional) to take off the gloves used with the patient. This message can remain on the display and/or the display can change a border or background color to provide a reminder of the mode of operation in which the touchless interface 22 is to be used.

Controller 24 can also serve a second purpose of actively detecting a change of mode. In a first configuration, mode can be automatically switched from non-sterile to sterile when the touchless interface 22 is first used, and then switched from sterile to non-sterile when the touch screen user input is first received at the touch screen interface 20. In a second configuration, user input specific to entering the sterile mode is required for the controller to enter the sterile mode. After this point, any use of the touch screen input on the touch screen interface 20 will cause the mode to switch to non-sterile. If the user accidentally forgets that he or she is in the sterile mode, at first touch of the screen of the touch screen interface 20 will cause the above-described message and/or color change on the display of the touch screen interface 20 to warn the user that gloves must now be changed before returning to the patient or to otherwise work in the sterile zone.

Controller 24 is illustrated in FIG. 1 as a component that is fully separate from controllers 14 and 16, however, as will be appreciated by those skilled in the art, controller 24 can be integrated with controllers 14 and 16. Displaying controls and detection of any user input or of specific user input (e.g. from a mode select control button provided on the display) involves components usually associated with components 14, 16, 20 and 22. Likewise, controllers 14 and 16 can be integrated into the same controls for both scanning and model building, if desired.

Although the present invention has been described herein above by way of specific embodiments thereof, it can be modified without departing from the spirit and nature of the subject invention as defined in the appended claims.

We claim:

1. An interface for controlling a medical device comprising a sterile mode of operation and a non-sterile mode of operation, the interface comprising:
a screen on which at least one control object for controlling the medical device is displayed;
a touch screen interface for manipulating said at least one control object; and
a touchless interface for manipulating said at least one control object;
wherein said touch screen interface is enabled when in the non-sterile mode of operation and disabled when in the sterile mode of operation.

2. The medical device interface of claim 1, further comprising a cursor displayed on said screen and controllable by said touch screen interface and said touchless interface for manipulating said at least one control object.

3. The medical device interface of claim 1, wherein said at least one control object comprises a graphical control element displayed on said screen.

4. The medical device interface of claim 1, wherein said graphical control element comprises one of a widget, a check box, a scroll bar, a radio button, a drop down box, a list box, a menu item, a button, spinner, a slider, a hyperlink and combinations thereof.

5. The medical device interface of claim 1, wherein said touchless interface is disabled when in said non-sterile mode of operation.

6. The medical device interface of claim 1, wherein when in the non-sterile mode of operation using said touchless interface activates the sterile mode of operation and when in the sterile mode of operation using said touchscreen interface activates the non-sterile mode of operation.

7. The medical device interface of claim 1, wherein when in the sterile mode of operation, using the touchscreen interface activates the non-sterile mode of operation and displays a warning to this effect on said screen.

8. The medical device interface of claim 1, wherein a plurality of control objects are displayed on said screen, wherein said touch screen interface can manipulate said plurality of control objects and wherein said touchless screen can manipulate a subset of said plurality of control objects.

9. The medical device interface of claim 1, wherein said touchless interface is disabled when in the non-sterile mode of operation.

10. The medical device interface of claim 1, wherein said touchless interface comprises a camera system for detecting gestures made in front of said screen.

11. The medical device interface of claim 1, wherein when in the sterile mode of operation, a warning to this effect is displayed on said screen.

12. The medical device interface of claim 11, wherein said warning comprises one of changing a border color of said screen, displaying a warning text on said screen, changing a background color of said screen and combinations thereof.

13. The medical device interface of claim 1, wherein said touchless interface comprises one of a foot operated mouse, foot operated trackball and combinations thereof.

14. The medical device interface of claim 1, wherein at least part of the medical device is moved between a sterile zone and a non-sterile zone, and wherein said sterile mode of operation is enabled when said part moves into said sterile zone and said non-sterile mode of operation is enabled when said part moves into said non-sterile zone.

15. The medical device interface of claim 1, wherein the medical device comprises a dental device comprising a handheld intraoral scanner and further wherein manipulating said control object activates said intraoral scanner.

16. A method for controlling a medical device comprising a screen, at least part of the medical device for use in both a sterile-zone and a non-sterile zone, the method comprising:
displaying at least one control object for controlling the medical device on the screen;
providing both a touch screen interface and touchless interface for manipulating said at least one control object;
disabling said touch screen interface when the part is within the sterile zone; and
enabling said touch screen interface when the part is within the non-sterile zone.

17. The method of claim 16, further comprising enabling said touchless interface when the part is within the sterile zone and disabling said touchless interface when the part is within the non-sterile zone.

18. The method of claim 16, further comprising displaying a cursor for manipulating said at least one control object on the screen and controlling said cursor using said touch screen interface and said touchless interface.

19. The method of claim 16, wherein said at least one control object comprises a graphical control element.

20. The method of claim 19, wherein said graphical control element comprises one of a widget, a check box, a scroll bar, a radio button, a drop down box, a list box, a menu item, a button, spinner, a slider, a hyperlink and combinations thereof.

21. A dental device comprising:
a handheld intraoral scanner comprising at least one camera;
a controller for sending commands to said controller and storing images captured by said handheld intraoral scanner;
a communication link between said handheld intraoral scanner and said controller for transmitting images captured by said intraoral scanner to said controller and transmitting commands from said controller to said handheld intraoral scanner;
a screen coupled to said controller and on which at least one control object is displayed;
a touch screen interface for manipulating said at least one control object; and
a touchless interface for manipulating said at least one control object;
wherein said touch screen interface is enabled when said in the non-sterile mode of operation and disabled when in the sterile mode of operation and further wherein said controller generates and sends said commands to said handheld intraoral scanner in response to manipulation of said control object by one of said touch screen interface, said touchless interface and combinations thereof.

22. The dental device of claim 21, wherein said commands comprise one of a command to start scanning and a command to stop scanning:
